(19) United States
(12) Patent Application Publication
(10) Pub. No.: US 2003/0233742 A1
(43) Pub. Date: Dec. 25, 2003

(54) Compressed absorbent tampon

(76) Inventors: Archie L. Jones, Somerset, NJ (US); Lai-Hing Louie, Kendall Park, NJ (US); Hien Nguyen, East Windsor, NJ (US); Judith E. Roller, North Brunswick, NJ (US); Edwin H. Sailer, Ringoes, NJ (US)

Correspondence Address: PHILIP S. JOHNSON, JOHNSON & JOHNSON, ONE JOHNSON & JOHNSON PLAZA, NEW BRUNSWICK, NJ, 08933-7003, US

(21) Appl. No.: 10/179,430

(22) Filed: Jun. 25, 2002

(51) Int. Cl. A61F 13/20

(52) U.S. Cl. 28/118

(57) ABSTRACT

A novel process for forming a compressed tampon includes the steps of forming an open structure comprising at least about 5 wt-% of cellulosic materials; heating the open structure to a temperature of at least about 40° C.; compressing the heated open structure to form the compressed tampon; and releasing the compressed fibrous tampon from compression. Surprisingly, both the force required for compression and the degree of over-compression are significantly reduced when the fibrous web is heated prior to compression. Indeed, what we have found is that heating the fibrous web prior to compression into the tampon form provides a more consistent, dimensionally-controlled product. It also requires lower compression forces to achieve a dimensionally stable product with reduced fiber damage.

### CROSS-REFERENCE TO RELATED APPLICATIONS

This invention is related to the following copending application: U.S. Ser. No. ------, filed Jun. 25, 2002, entitled “Compressed Absorbent Web” (Att'y Docket, PPC-842).

1. Field of the Invention

The present invention relates to a process and apparatus for forming a densified structure using relatively low compression forces. The process includes heating an open structure prior to compression, and the apparatus includes elements for this heating.

2. Background of the Invention

Absorbent structures are manufactured under compression to provide sufficient absorbent capacity for a given use in a conveniently dimensioned product. Absorbent structures may include wound care, diapers, sanitary napkins, tampons, and interlabial devices.

Many absorbent structures, such as tampons, achieve shape stability by slightly overcompressing the structure and allowing it to recover or expand to the desired dimensions. This structure may also be heat set. An example of this is described in Johst et al., U.S. Pat. No. 4,081,884. This patent discloses radially compressing a tampon blank comprising cellulosic fibers, introducing the radially compressed tampon blank into a heated chamber, and axially compressing the tampon while heating for at least about five seconds. This process requires significant time to set the tampon.

Another example is disclosed in Wollangk et al., U.S. Pat. No. 4,326,527, which purports to disclose a process for rapidly and uniformly heat-setting a radially compressed tampon. The process includes the steps of compressing a prehumidified tampon and subjecting the compressed tampon to microwave heating while the tampon is in an open ended tube having openings about the longitudinal axis of the tube to heat-set the tampon. This process requires significant energy to set the tampon and the prehumidification may promote the growth of undesirable microorganisms during the manufacture of the tampon.

While the amount of compressive energy required in these references is not discussed in any great detail, it is our experience that the energy required to radially compress a commercial tampon, as measured by the compressive force, is very great. This is especially true for non-conventional fiber blends, such as those containing non-cellulosic materials. This energy requirement can also limit the ability to commercially produce tampons having increased density. High compressive forces can damage process equipment and negatively affect the tampon performance by damaging the fibers within the tampon structure. This fiber damage can lead to poor expansion and absorbent capacity of the product.

Therefore, what is needed is a process for forming a compressed tampon that employs lower compressive force to reduce the risk of web and equipment damage.

### SUMMARY OF THE INVENTION

It is an object of the present invention to provide process that can provide a compressed absorbent tampon web using lower compressive force than would otherwise be required to reduce the risk of web and equipment damage. This can produce a tampon having low density relaxation (as defined in the Examples).

It is another object of the present invention to provide a tampon having low density relaxation and that has a low degree of fiber damage despite being sufficiently compressed to form a tampon having an appropriate density.

In accordance with the present invention, there has been provided a novel process for forming a compressed tampon. The process includes the steps of forming an open structure comprising at least about 5 wt-% of cellulosic materials; heating the open structure to a temperature of at least about 40° C.; compressing the heated open structure to form the compressed tampon; and releasing the compressed fibrous tampon from compression. Surprisingly, both the force required for compression and the degree of over-compression are significantly reduced when the fibrous web is heated prior to compression. Indeed, what we have found is that heating the fibrous web prior to compression into the tampon form provides a more consistent, dimensionally-controlled product. It also requires lower compression forces to achieve a dimensionally stable product with reduced fiber damage.

### BRIEF DESCRIPTION OF THE DRAWING

FIG. 1 is a perspective view of a tampon of the present invention.

FIG. 2 is a diagrammatic view of an apparatus for producing a tampon according to one embodiment of the present invention.

FIG. 2A is a cross-section of carrier having a substantially cylindrical carrier for use in a modification of the apparatus of FIG. 2.

FIG. 3 is a diagrammatic view of an apparatus for producing a tampon according to another embodiment of the present invention.

### DETAILED DESCRIPTION OF THE PREFERRED EMBODIMENTS

As used in the specification and claims, the term “open structure” and variations of this term relate to compressible structures prior to substantial compression to form compressed absorbent products, such as tampons. For example, these open structures may be formed by carding, air laying, or other processes and may include some minor calendering to maintain a density of less than about 0.1 g/cm³.

As used in the specification and claims, the term “compressible” and variations of this term relates to structures that can be compressed to hold a generally compressed form and that also can expand to a relatively uncompressed state upon exposure to sufficient moisture or liquid.

As used in the specification and claims, the term “radially expand” and variations of this term relate to the expansion of elongate tampons. These tampons expand primarily in a direction perpendicular to the central axis of the tampon. Preferably, the tampons expand in at least one direction perpendicular to the central axis, more preferably, at least two directions. Most preferably, the tampons expand substantially uniformly in all directions perpendicular to the central axis.

As used in the specification and claims, the term “axially expand” and variations of this term relates to the expansion of another particular class of elongate tampons. These tampons expand primarily in a direction along the central axis of the tampon. However, the tampons may also expand in at least one other direction.

The absorbent tampons of the present invention elongate masses of compressed materials, preferably substantially cylindrical masses of compressed materials having a central axis and a radius that defines the outer circumferential surface of the tampon. Tampons are often formed by first obtaining a shaped mass of materials called a tampon blank. This blank can be in the form of a roll of a nonwoven web, a mass of randomly or substantially uniformly oriented material, and the like.

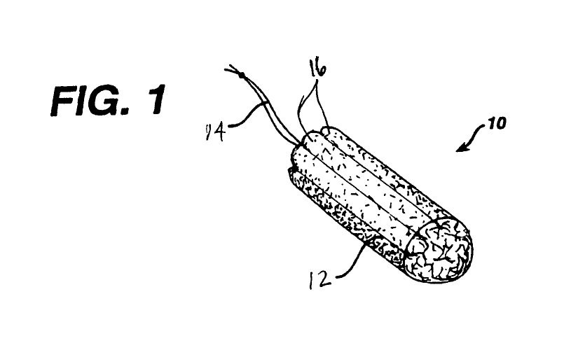

The tampon blank is an open structure that is relatively uncompressed and has a relatively low density. It is then compressed to form a product having smaller dimensions and a higher density than the tampon blank. After the tampon is released from compression, it relaxes (or expands), slightly, to its final dimensions. The compressed tampons may have a generally uniform density throughout the tampon, or they may have regions of differing density as described in the commonly assigned patents to Friese et al., U.S. Pat. No. 6,310,269, and Leutwyler et al., U.S. Pat. No. 5,911,712, the disclosures of which are herein incorporated by reference. As shown in FIG. 1, tampons 10 also usually include a cover 12 or some other surface treatment and a withdrawal string 14 or other removal mechanism.

The tampon 10 may have a relatively dense core substantially surrounding its central axis and a less dense annulus surrounding the core and forming the outer circumferential surface. This density differential may be provided by relatively uniform, yet distinct, absorbent material distribution within the core and annulus, or it may be provided by a plurality of ribs 16 which extend radially from the core.

The materials that may be used in the tampon include fibers, foams, and particles or other discrete materials. The tampon include cellulosic fibers. A useful, non-limiting list of useful cellulosic fibers includes natural fibers such as cotton, wood pulp, jute, hemp, sphagnum, and the like; and processed materials including cellulose derivatives such as regenerated cellulose (including rayon and lyocell), cellulose nitrate, carboxymethyl cellulose, and the like. The tampons may also include other materials including, without limitation, polyester, polyvinyl alcohol, polyolefin, polyamine, polyamide, polyacrylonitrile, and the like.

Preferably, the tampons are formed predominantly of fibers. The fibers may be any of the materials listed above, and may have any useful cross-section, including multi-limbed and non-limbed. Multi-limbed, regenerated cellulosic fibers have been commercially available for a number of years. These fibers are known to possess increased specific absorbency over non-limbed fibers. Commercial examples of these fibers are Danufil VY trilobal viscose rayon fibers available from Acordis Ltd., Spondon, England. These fibers are described in detail in Wilkes et al, U.S. Pat. No. 5,458,835, the disclosure of which is hereby incorporated by reference.

Preferably, the tampon includes at least about 5 wt-% of the cellulosic materials. These materials are moisture sensitive, and provide hydrogen bonding when compressed under moist conditions. More preferably, the tampon includes about 35 to about 100 wt-% cellulosic materials, and most preferably, about 50 to about 75 wt-% cellulosic materials.

Prior to heating, the open structure has a moisture content of at least about 4 wt-%, preferably, about 8 to about 13 wt-%. After heating, the open structure retains sufficient moisture content to promote interfiber bonds sufficient to maintain the dimensions of the compressed tampon. Preferably, the open structure a moisture content of about 2 to about 13 wt-% after heating.

Preferably, the tampon blank is substantially enclosed by a fluid-permeable cover. Thus, the cover encloses a majority of the outer surface of the tampon. This may be achieved as disclosed in Friese, U.S. Pat. No. 4,816,100, the disclosure of which is herein incorporated by reference. In addition, either or both ends of the tampon may be enclosed by the cover. Of course, for processing or other reasons, some portions of the surface of the tampon may be free of the cover. For example, the insertion end of the tampon and a portion of the cylindrical surface adjacent this end may be exposed, without the cover to allow the tampon to more readily accept fluids.

The cover can ease the insertion of the tampon into the body cavity and can reduce the possibility of fibers being separated from the tampon. Those of ordinary skill in the art will recognize covers that are useful in conjunction with the tampons of the present invention. They may be selected from an outer layer of fibers which are fused together (such as by thermobonding), a nonwoven fabric, an apertured film, or the like.

Tampons are generally categorized in two classes: applicator tampons and digital tampons, and a certain amount of dimensional stability is useful for each type of tampon. Applicator tampons use a relatively rigid device to contain and protect the tampon prior to use. To insert the tampon into a body cavity, the applicator is partially inserted into the body cavity, and the tampon can be expelled therefrom. In contrast, digital tampons do not have an applicator to help guide them into the body cavity and require sufficient column strength to allow insertion without using an applicator. This strength can be determined by securing one end of the tampon to the fixed plate of a Instron Universal Testing Machine, available from Instron Corporation, Canton, Mass., U.S.A. The moveable plate is brought to contact the opposite end of the tampon and is then set to compress the tampon at a rate of about 5 cm/minute. The force exerted on the tampon is measured continuously, and the point at which this force begins to fall instead of rise is the point at which the tampon buckles. The maximum force achieved is the tampon stability. Preferably, digital tampons of the present invention have a significant stability, at least about 10 N. More preferably, the digital tampons have a stability of at least about 20 N, and most preferably, they have a stability of about 30 N to about 85 N. Tampons with a stability that is too low do not have sufficient dimensional stability to maintain their basic structure during insertion as a digital tampon; tampons with a stability which is too high can be perceived as being too stiff or hard to be comfortably inserted as a digital tampon.

While the applicator tampon is protected by the rigid applicator device and the applicator tampon need not as have high a degree of column strength as a digital tampon, applicator tampons do require dimensional stability (especially radial) to be acceptable for use. This dimensional stability provides assurance, for example, that the tampon will not prematurely grow and split its packaging material or become wedged in a tampon applicator.

The process of the present invention begins with an open structure. The open structure may be a nonwoven web, a mass of randomly or substantially uniformly oriented materials, such as fibers, foams, or particles, and the like. This mass is then manipulated to form a tampon blank.

A nonwoven web useful in the present invention can be formed in any manner desired by the person of ordinary skill in the art. For example, fibers can be opened and/or blended by continuously metering them into a saw-tooth opener. The blended fibers can be transported, e.g., by air through a conduit to a carding station to form a fibrous web. Alternatively, a mass of substantially randomly oriented fibers can be formed by opening and/or blending them, transporting them, as above, to a station to form, e.g., a teabag-type tampon blank. Further processes may employ oriented fibers in a fibrous tow.

The tampon blank can be further processed to form a tampon. In a tampon forming process, a web can be formed into a narrow, fibrous sliver and spirally wound to form a tampon blank. In addition, a liquid-permeable cover material can be wrapped around the tampon blank to substantially contain the fibrous absorbent portion of the tampon. Examples of the further processing of the webs are described in Friese et al., U.S. Pat. No. 4,816,100, and Schwankhardt, U.S. Pat. No. 5,909,884 (the disclosures of which are herein incorporated by reference). However, these processes are to be modified according to the present invention.

The open structure may be heated prior to its formation into a tampon blank. The open structure may also be heated after its formation into a tampon blank, or even both before and after. The resulting pre-heated tampon blank can then be compressed at a significantly reduced pressure to form a dimensionally stable tampon.

In the process of Friese et al., a fibrous web 100, having a width corresponding to the length of the tampon 10, is supplied continuously and is weakened transversely to its longitudinal direction. This weakening may be achieved through perforating and stretching of the web to reduce its cross-section at a weakened zone. A continuously supplied cover strip is severed to form a cover section 102, the length of which exceeds the circumference of the tampon blank 104 as shown in the winding station 106. The cover section 102 is bonded (e.g., thermally) sealed to the outside of a region of the web 100 at one end of the web section adjacent the weakened zone. The cover section 102 is arranged on the web 100 such that a free end 102a of the cover section 102 extends beyond the weakened zone. The web 100 can then be severed at the weakened zone to form a gap 108 between adjacent web sections 110.

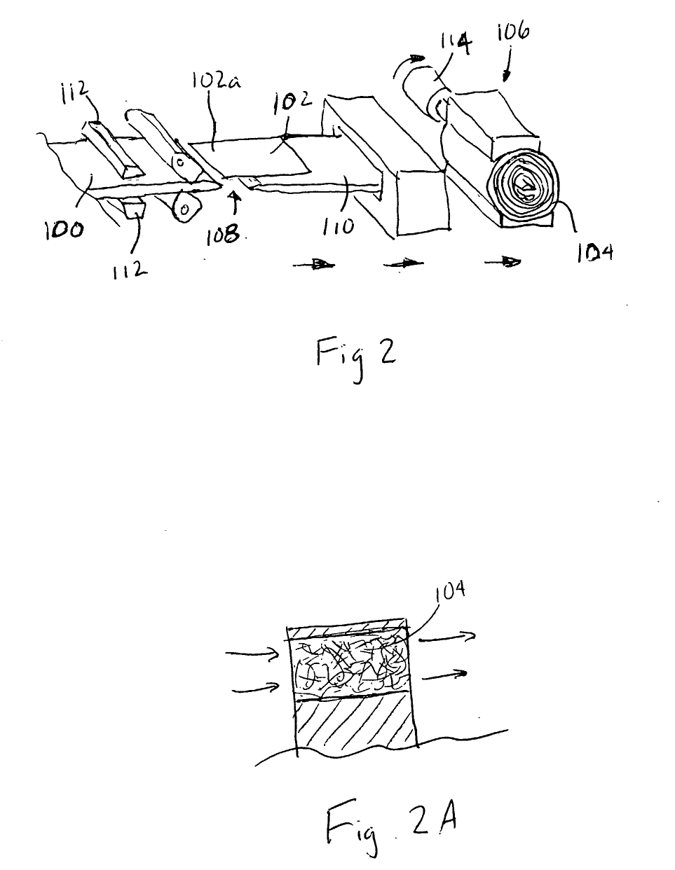

The web 100 can be heated by means of heaters 112, which may, e.g., be placed prior to the severing of adjacent web sections 110. A heated web sections 110 can then be wound upon itself about an axis extending transversely to its longitudinal direction by a winding mandrel 114. This forms a tampon blank 104. The wound-up tampon blank 104 can efficiently retain the applied heat due to an insulating effect of the outer layers of the web. This may be enhanced by enclosing the process equipment around the heated tampon blank and/or heated web sections. Alternatively, heated air can be forced through the relatively loosely wound-up tampon blank 104 to preheat the blank prior to compression as shown in FIG. 2A.

Heat can be applied to the fibrous mass or web via conduction, convection, radiation, combinations of these, and the like. Such processes include, without limitation, circulation of hot air or steam, electromagnetic transmission of energy (for example, without limitation, radio frequency energy, infrared energy, microwave energy, and the like), insertion of heated pins into web to provide conductive heat transfer, ultrasonic energy, and the like. The open structure is heated to a temperature of at least about 40° C. More preferably, the open structure is heated to at least about 45° C., and most preferably, the open structure is heated to at least about 60° C. In order to avoid over-heating some thermoplastic fibers or over-drying the structure, it may also be beneficial to limit the temperature of the open structure to less than about 100° C. or even 85° C.

After winding, the cover section 102 completely surrounds the circumference of the tampon blank 104 over the intended width, and the free end 102a can be thermally bonded to an overlapped portion of itself on the outside of the tampon blank 104.

A withdrawal string can be placed around the web section 110 prior to winding and, if appropriate, knotted at its free ends. The finished tampon blank 104 can then be delivered to a tampon press, as is disclosed in Leutwyler et al., U.S. Pat. No. 5,911,712.

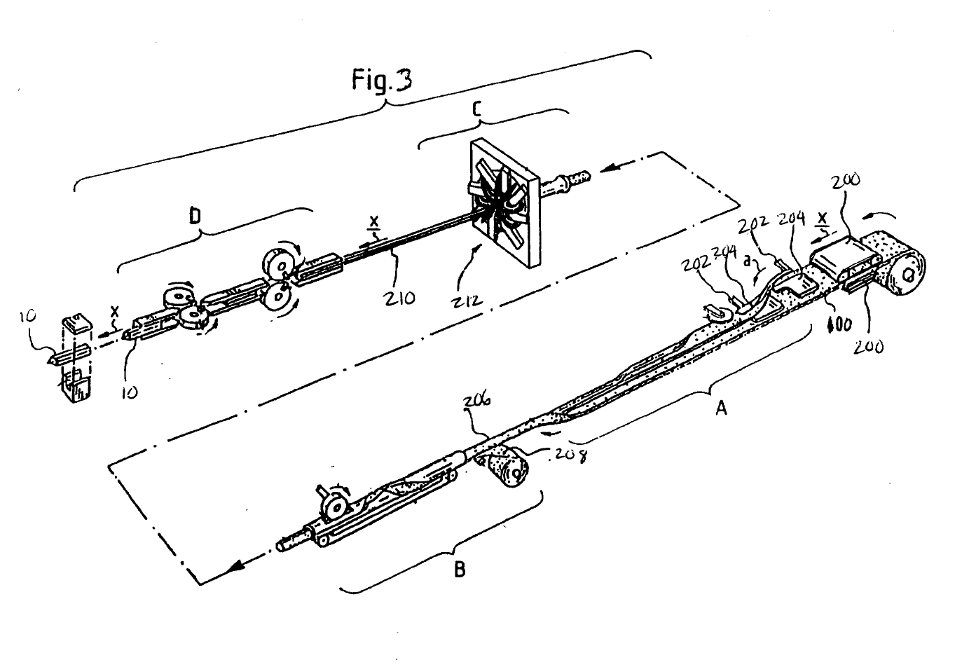

As shown in FIG. 3, coincident with the process of Schwankhardt, a fibrous web 100 can be heated by a heater 200 prior to entering the folding station A in which a series of folding plates 202 and baffle plates 204 sequentially fold the web to form a folded or essentially wound-up fibrous rope 206 as it exits the folding station. The fibrous rope 206 can be enveloped in a cover material 208 (a wrapping band according to Schwankhardt) in a wrapping station B, compressed into a compressed strand 210 in a press 212 of a press station C, and cut and formed into individual tampons 10 in a severing station D, and packaged. The heating is preferably performed prior to the first folding plate 202 and baffle plate 204 to allow for substantially uniform heating through the thickness of the web 100. However, the heating can be performed or supplemented further into the folding station, preferably before too many layers of the web are folded up.

In addition to the processes described above, the processes of manufacturing tampons disclosed in Haas, U.S. Pat. No. 1,926,900, Voss, U.S. Pat. No. 2,076,389, and Dostal, U.S. Pat. No. 3,811,445 (the disclosures of which are herein incorporated by references), can be modified in similar fashions to that described above to take advantage of the inventive concepts herein disclosed.

As described above, tampons are generally over-compressed to mechanically constrict spontaneous expansion of the structure thereby preventing the tampon from expanding too much before use. However, this over-compression is not always uniform, and its effectiveness varies. In particular, when large masses of fibers are compressed, localized volumes may be subjected to greater compressive forces than other volumes. This may be desirable as in the Friese and Leutwyler disclosures, described above. Unfortunately, it can also result in compression being concentrated in the outer regions of a tampon and result in less control of the dimensional stability of the tampon.

Surprisingly, both the force required for compression and degree of over- compression are significantly reduced when the fibrous web is heated prior to compression. This is also reflected in the lower density relaxation. Based upon these findings, we expect that heating the fibrous web prior to compression into the tampon form provides a more consistent, dimensionally-controlled product. It also requires lower compression forces to achieve a dimensionally stable product.

One way to illustrate the consistency and dimensional control of the tampon is a review of the density relaxation (as defined below in the Examples) of the tampon. Preferably, the tampons of the present invention have a density relaxation of less than about 20%, more preferably, less than about 10%, and most preferably, less than about 5%.

Another way to illustrate the advantages of the present invention is the reduced fiber damage in the tampon. Fiber damage, including permanent fiber deformation and breakage, occurs during compression of the tampon. Fiber damage can be determined by examining the tampon for fibers that have been broken. For example, tampons that are formed of staple length fibers (about 1 to 1.5 inches (25 to about 40 mm)) can be inspected to determine the number or percentage of fibers that have a length of less than about ¾ inch (18 mm). Alternatively, these tampons can be analyzed to determine the percentage of fines (fibers having a length of less than about ¼ inch (7 mm)). A significant percentage of short fibers or fines can be indicative of fiber damage in a product.

After heating, the open structure and/or tampon blank beneficially retains its heat due to the inherent insulating properties of a loosely gathered mass of fibers and the heated air trapped in the capillaries thereof. Looser capillaries of the more open web allow more even heat conduction into center of the web. Further, when the unrolled web is heated there is less thermal mass between the heat source and the center of the web than there is when the heat is provided to the compressed tampon. Of course, the applied heat can dissipate into the atmosphere if compression does not follow the heating within a reasonable time.

While we have found that the above process can provide sufficient dimensional stability without additional post-compression heating, some amount of post-compression heating may be desirable to optimize a manufacturing process. However, any such post-compression heat required is greatly reduced over prior art processes which do not employ any pre-compression heating.

We also believe that the present process allows for increased manufacturing line speeds and improved processability. For example, we have found that early fiber heating decreases the amount of materials, such as fibers, lost from the open structure during pre-compression handling. This produces a more consistent material stream leading into later processing stations.

### EXAMPLES

The present invention will be further understood by reference to the following specific Examples that are illustrative of the composition, form and method of producing the device of the present invention. It is to be understood that many variations of composition, form and method of producing the device would be apparent to those skilled in the art. The following Examples, wherein parts and percentages are by weight unless otherwise indicated, are only illustrative.

### Example 1

A blend of 75 wt-% 3 denier Danufil® VY trilobal viscose rayon fibers and 25 wt-% 3 denier Danufil® V viscose rayon fibers, both available from Acordis Ltd., Spondon, England, were opened via standard fiber opening and carding equipment. A fixed amount of the fiber blend (having a mass, W, of about 2 g) was introduced in a stainless steel mold with a cylindrical cavity (of cross-sectional area, A, of about 5 cm²). A cylindrical plunger size matched to the cylindrical cavity was used to compress the fiber mass using a standard laboratory press. In order to heat the samples, the mold and plunger were heated together in an oven set at the target temperature. After sufficient time to allow the mold and plunger to reach the oven temperature, the fibers were placed into the cavity, and the mold, plunger and fibers were heated for an additional three minutes to allow the fibers to reach the oven temperature. The heated assembly was removed from the oven and placed between the plates of the laboratary press. Pressure was applied to compress the fiber mass in the cavity up to a predetermined peak pressure and released, after which the compressed fibrous plug was removed to allow an immediate measurement of the initial thickness, T₀.

The compressed fibrous plug had an initial volume (V₀=A * T₀) and an initial density (ρ=W/V₀), but the plug expanded after the pressure was released, reaching equilibrium after about 15 to 20 minutes (at room temperature, approximately 20° C.). While the humidity conditions of the test are not generally critical, conducting the test at high humidity will adversely affect the test results. The equilibrium thickness, Tₑ, was then measured to provide an equilibrium density (ρₑ=W/(A * Tₑ)). From these values, a “Density relaxation” can be calculated equal to (ρ₀−ρₑ)/ ρ₀.

A control was also prepared employing mold, plunger and fibers at room temperature, about 20° C. The results of measurements at each temperature and pressure are shown in Table 1.

TABLE 1

| | | | Equilibrium | % Density |
|---|---|---|---|---|
| Temperature | Peak Pressure | Initial Density | Density | relaxation |
| 100° C. | 610 | 0.46 | 0.45 | 2% |
| | 1200 | 0.62 | 0.62 | <2% |
| | 1800 | 0.75 | 0.75 | <2% |
| | 2500 | 0.80 | 0.80 | <2% |
| | 3000 | 0.85 | 0.84 | <2% |
| | 3600 | 0.88 | 0.88 | <2% |
| | 4800 | 0.92 | 0.92 | <2% |
| | 6100 | 0.95 | 0.95 | <2% |
| 85° C. | 610 | 0.34 | 0.34 | <2% |
| | 910 | 0.49 | 0.49 | <2% |
| | 1200 | 0.43 | 0.43 | <2% |
| | 1500 | 0.55 | 0.55 | <2% |
| | 1800 | 0.53 | 0.53 | <2% |
| | 3600 | 0.85 | 0.86 | <2% |
| | 4900 | 0.85 | 0.86 | <2% |
| 75° C. | 610 | 0.36 | 0.36 | <2% |
| | 910 | 0.40 | 0.39 | 3% |
| | 1200 | 0.52 | 0.51 | 2% |
| | 1500 | 0.54 | 0.53 | <2% |
| | 1800 | 0.65 | 0.64 | <2% |
| | 3600 | 0.80 | 0.80 | <2% |
| | 4900 | 0.84 | 0.84 | <2% |
| | 6100 | 0.91 | 0.92 | <2% |
| 60° C. | 910 | 0.33 | 0.32 | 3% |
| | 1200 | 0.41 | 0.40 | 2% |
| | 1500 | 0.44 | 0.44 | <2% |
| | 1800 | 0.49 | 0.47 | 4% |
| | 2200 | 0.57 | 0.55 | 4% |
| | 3000 | 0.65 | 0.64 | <2% |
| | 4900 | 0.79 | 0.78 | <2% |
| | 6100 | 0.87 | 0.87 | <2% |
| 40° C. | 1200 | 0.31 | 0.30 | 3% |
| | 1800 | 0.43 | 0.40 | 7% |
| | 2400 | 0.50 | 0.47 | 6% |
| | 3000 | 0.58 | 0.56 | 3% |
| | 4300 | 0.69 | 0.66 | 4% |
| ROOM | 1200 | 0.23 | 0.17 | 26% |
| | 2400 | 0.33 | 0.25 | 24% |
| | 3600 | 0.50 | 0.38 | 24% |
| | 4900 | 0.60 | 0.46 | 23% |
| | 6200 | 0.64 | 0.51 | 20% |

These data show that pre-heating of fibers at a temperature of at least about 40° C. and maintaining the heat during compression provides a significantly greater dimensional stability than compressing the same fibers at room temperature. They further illustrate that substantially higher fiber plug densities can be achieved at lower compression pressures when fibers are pre-heated. This is even more pronounced at temperatures of greater than about 60° C.

### Example 2

The procedure of Example 1 was repeated with a blend of 75 wt-% 3 denier Danufil® V viscose rayon fibers, available from Acordis Ltd. (Spondon, England), and 25 wt-% 3 denier T-224 polyester fibers, available from KoSa, (Houston, Tex., USA). Again, the results of measurements at each temperature and pressure are shown in Table 2.

TABLE 2

| | | | Equilibrium | % Density |
|---|---|---|---|---|
| Temperature | Peak Pressure | Initial Density | Density | relaxation |
| 100° C. | 610 | 0.33 | 0.34 | <2% |
| | 1200 | 0.47 | 0.48 | <2% |
| | 7400 | 0.62 | 0.63 | <2% |
| | 3000 | 0.65 | 0.65 | <2% |
| 85° C. | 910 | 0.36 | 0.35 | 3% |
| | 1200 | 0.39 | 0.39 | <2% |
| | 2400 | 0.53 | 0.53 | <2% |
| | 3600 | 0.66 | 0.66 | <2% |
| | 4900 | 0.80 | 0.79 | <2% |
| | 6100 | 0.79 | 0.78 | <2% |
| 75° C. | 910 | 0.33 | 0.32 | 3% |
| | 1200 | 0.37 | 0.37 | <2% |
| | 2400 | 0.52 | 0.50 | 4% |
| | 3600 | 0.65 | 0.64 | <2% |
| | 4900 | 0.7T | 0.70 | <2% |
| | 6100 | 0.79 | 0.80 | <2% |
| 60° C. | 610 | 0.25 | 0.24 | 4% |
| | 1200 | 0.38 | 0.37 | 3% |
| | 2400 | 0.50 | 0.50 | <2% |
| | 3600 | 0.62 | 0.60 | 3% |
| | 6100 | 0.77 | 0.75 | 3% |
| 45° C. | 910 | 0.30 | 0.29 | 3% |
| | 1200 | 0.34 | 0.34 | <2% |
| | 2400 | 0.44 | 0.43 | 2% |
| | 3600 | 0.59 | 0.59 | <2% |
| | 4900 | 0.71 | 0.69 | 3% |
| | 6100 | 0.77 | 0.76 | <2% |
| ROOM | 2400 | 0.41 | 0.33 | 20% |
| | 3600 | 0.51 | 0.40 | 22% |
| | 3800 | 0.55 | 0.40 | 27% |
| | 3800 | 0.52 | 0.40 | 23% |
| | 4900 | 0.61 | 0.52 | 15% |

These data show that pre-heating of fibers at a temperature of at least about 45° C. and maintaining the heat during compression provides a significantly greater dimensional stability than compressing the same fibers at room temperature. They further illustrate substantially higher fiber plug densities can be achieved at lower compression pressure when fibers are pre-heated. This is even more pronounced at temperature of greater than about 60° C. However, with thermoplastic fibers, such as polyester fiber, this preheating may be limited to avoid exceeding their yield point to cause permanent deformation of the fibers, including melting the fibers.

### Example 3

The procedure of Example 1 was repeated with different blends of 3 denier Danufil® V viscose rayon fibers, available from Acordis Ltd. (Spondon, England), and 3 denier T-224 polyester fibers, available from KoSa, (Houston, Tex., USA). However, in this series, the temperature was maintained at 75° C., while the proportion of fibers varied. The results of measurements at each blend and pressure are shown in Table 3.

TABLE 3

| | | | Equilibrium | % Density |
|---|---|---|---|---|
| | Peak Pressure | Initial Density | Density | relaxation |
| 25% PET | 910 | 0.33 | 0.32 | 3% |
| | 1200 | 0.37 | 0.37 | <2% |
| | 2400 | 0.52 | 0.50 | 4% |
| | 3600 | 0.65 | 0.64 | <2% |
| | 4900 | 0.71 | 0.70 | <2% |
| | 6100 | 0.79 | 0.80 | <2% |
| 33% PET | 610 | 0.24 | 0.23 | 4% |
| | 910 | 0.31 | 0.30 | 3% |
| | 1200 | 0.39 | 0.38 | 3% |
| | 2400 | 0.49 | 0.47 | 4% |
| 50% PET | 910 | 0.28 | 0.28 | <2% |
| | 1200 | 0.32 | 0.31 | 3% |
| | 2400 | 0.49 | 0.47 | 4% |
| | 3600 | 0.59 | 0.57 | 3% |
| | 4900 | 0.68 | 0.67 | <2% |
| | 6100 | 0.75 | 0.75 | <2% |
| 67% PET | 1200 | 0.31 | 0.30 | 3% |
| | 2400 | 0.41 | 0.41 | <2% |
| | 3600 | 0.63 | 0.62 | <2% |
| | 6100 | 0.70 | 0.69 | <2% |

This data show that pre-heating of fibers at a temperature of about 75° C. and maintaining the heat during compression provides significant dimensional stability, even with large proportions of relatively resilient fibers, such as polyester.

The specification and embodiments above are presented to aid in the complete and non-limiting understanding of the invention disclosed herein. Since many variations and embodiments of the invention can be made without departing from its spirit and scope, the invention resides in the claims hereinafter appended.

What is claimed is:

1. A process for forming a compressed tampon comprising the steps of:
forming an open structure comprising at least about 5 wt-% of cellulosic materials;
heating the open structure to a temperature of at least about 40° C., while substantially maintaining moisture present in the open structure, to form a heated open structure;
compressing the heated open structure to form the compressed tampon; and
releasing the compressed tampon from compression.

2. The process of claim 1 wherein the open structure comprises at least about 15 wt-% of cellulosic materials.

3. The process of claim 1 wherein the open structure further comprises at least about 5 wt-% non-cellulosic polymeric materials.

4. The process of claim 1 comprising heating the open structure to a temperature of at least about 45° C.

5. The process of claim 4 comprising heating the open structure to a temperature of at least about 60° C.

6. The process of claim 1 wherein the step of heating the open structure comprises maintaining at least about 2 wt-% moisture in the open structure.

7. The process of claim 1 which further comprises the step of forming the open structure into an elongate shape.

8. The process of claim 7 wherein the open structure is heated prior to forming the open structure into an elongate shape.

9. The process of claim 7 wherein the open structure is formed into an elongate shape prior to heating.

10. The process of claim 7 wherein the open structure is a fibrous web, and the step of forming the open structure into an elongate shape comprises winding the fibrous web to form a substantially cylindrical shape.

11. The process of claim 1 comprising the step of compressing the heated open structure to form the compressed tampon to a density of greater than about 0.3 g/cm³.

12. The process of claim 11 comprising the step of compressing the heated open structure to form the compressed tampon to a density of greater than about 0.4 g/cm³.

13. The process of claim 1 wherein the cellulosic materials comprise cellulosic fibers.

14. The process of claim 3 wherein the non-cellulosic polymeric materials comprise fibers.

15. The process of claim 1 wherein the step of heating the open structure comprises moving heated air through the open structure.

16. The process of claim 15 wherein the heated air is humidified.

17. The process of claim 1 wherein the step of heating the open structure comprises applying electromagnetic energy to the open structure.

18. The process of claim 17 wherein the electromagnetic energy is selected from the group consisting of infrared radiation, ultrasonic energy, and microwave energy.

19. The process of claim 1 further comprising the step of enclosing the compressed tampon in packaging material.

20. The process of claim 1 wherein the compressed tampon has a density relaxation of less than about 20%.

21. The process of claim 20 wherein the compressed tampon has a density relaxation of less than about 10%.

22. A process for forming a compressed fibrous tampon comprising the steps of:
forming an open fibrous web comprising at least about 25 wt-% of cellulosic fibers;
heating the open fibrous web to a temperature of at least about 45° C., while substantially maintaining moisture present in the open fibrous web, to form a heated open fibrous web;
forming and compressing the heated open fibrous web to form an elongate fibrous tampon;
releasing the fibrous tampon from compression.

23. The process of claim 22 wherein the open fibrous web further comprises as least about 5 wt-% non-cellulosic polymeric materials.

24. The process of claim 22 comprising heating the open fibrous web to a temperature of at least about 60° C.

25. The process of claim 22 wherein the step of heating the open fibrous web comprises substantially maintaining moisture present in the open structure.

26. The process of claim 25 wherein the step of heating the open fibrous web comprises maintaining at least about 2 wt-% moisture in the open structure.

27. The process of claim 22 wherein the step of heating the open fibrous web comprises moving heated air through the fibrous web.

28. The process of claim 22 wherein the step of heating the open fibrous web comprises transmitting electromagnetic energy to the open fibrous web.

29. The process of claim 22 comprising the step of compressing the heated open fibrous web to form the elongate fibrous tampon to a density of greater than about 0.3 g/cm³.

30. The process of claim 29 comprising the step of compressing the heated open fibrous web to form the elongate fibrous tampon to a density of greater than about 0.4 g/cm³.

31. The process of claim 22 wherein the elongate tampon has a density relaxation of less than about 20%.

32. The process of claim 31 wherein the elongate tampon has a density relaxation of less than about 10%.
